(19) United States
(12) Patent Application Publication
(10) Pub. No.: US 2024/0124023 A1
(43) Pub. Date: Apr. 18, 2024

(54) VEHICULAR COMMUNICATION SYSTEM FOR AUTOMATIC COMMUNICATION WITH EMERGENCY VEHICLES AND HOSPITALS

(71) Applicant: Magna Electronics Inc., Auburn Hills, MI (US)

(72) Inventors: Prashanth Jay Kumar, Bengaluru (IN); Jagadish Narayan Gowda, Bengaluru (IN)

(21) Appl. No.: 18/484,558

(22) Filed: Oct. 11, 2023

Related U.S. Application Data
(60) Provisional application No. 63/379,315, filed on Oct. 13, 2022.

(51) Int. Cl. B60W 60/00; G16H 80/00

(52) U.S. Cl. CPC B60W 60/0015; G16H 80/00; B60W 2540/221; B60W 2556/45

(57) ABSTRACT

A vehicular control system includes an electronic control unit (ECU) disposed at a vehicle. The system is in wireless communication with a remote server. A health monitoring sensor captures sensor data representative of a health condition of an occupant of the vehicle. The system, based on processing at the ECU of captured sensor data, transmits a first signal to the remote server based on the captured sensor data. Responsive to receiving the first signal, the remote server transmits a second signal to the system including at least one destination suitable to treat the health condition of the occupant. Based on the second signal, the system selects a target destination from the at least one destination and determines a path of travel between the vehicle's current geographic location and the target destination's geographic location. The system at least partially controls operation of the vehicle along the path of travel.

### CROSS REFERENCE TO RELATED APPLICATION

The present application claims the filing benefits of U.S. provisional application Ser. No. 63/379,315, filed Oct. 13, 2022, which is hereby incorporated herein by reference in its entirety.

### FIELD OF THE INVENTION

The present invention relates generally to a vehicular communication system for a vehicle and, more particularly, to a vehicular communication system that communicates with systems remote from the vehicle.

### BACKGROUND OF THE INVENTION

It is known to provide a navigation system at a vehicle that provides directions for travel of the vehicle from a current location to a target destination.

### SUMMARY OF THE INVENTION

A vehicular communication system includes an electronic control unit (ECU) that includes electronic circuitry and associated software. The electronic circuitry includes a wireless communication module. The wireless communication module is configured to wirelessly communicate with a remote server disposed remote from a vehicle equipped with the vehicular communication system. For example, the remote server may be associated with one or more emergency services facilities, or systems or locations. One or more health monitoring sensors are disposed in the vehicle and configured to capture health data related to a person being transported by the vehicle. The vehicular communication system, via the wireless communication module, communicates captured health data to the remote server. Based on the communicated captured health data, the remote server communicates a destination waypoint to the communication system. The communication system provides the destination waypoint to a driving assist system of the vehicle and the driving assist system at least partially (such as autonomously or semi-autonomously) controls operation of the vehicle as the vehicle travels along a road toward a geographic location associated with the destination waypoint.

Optionally, the vehicle may be equipped with a vision system including one or more cameras or sensors disposed at the vehicle and the ECU may include an image processor or data processor configured to process sensor data captured by the one or more sensors, such as to enable the driving assist system to at least partially control the vehicle as the vehicle travels toward the destination waypoint.

These and other objects, advantages, purposes and features of the present invention will become apparent upon review of the following specification in conjunction with the drawings.

### BRIEF DESCRIPTION OF THE DRAWINGS

FIG. 1 is a plan view of a vehicle equipped with a communication system; and

FIG. 2 is a schematic view of the communication system.

### DESCRIPTION OF THE PREFERRED EMBODIMENTS

A vehicular communication system and/or driver or driving assist system operates to wirelessly communicate with an external or remote server remote from a vehicle equipped with the communication system. The vehicle may comprise an emergency vehicle (such as an ambulance, police vehicle, fire truck, or other emergency services vehicle) and the system wirelessly communicates patient health data related to the health of a victim or patient being transported in the emergency vehicle to the remote server. The patient health data may be captured by health monitors or sensors disposed in the emergency vehicle and in communication with the communication system. Based on the patient health data, the remote server communicates with the system at the emergency vehicle, such as to direct the emergency vehicle to an emergency services location (e.g., a particular hospital that can best treat the patient or that has availability or capacity to treat the patient). Optionally, the emergency vehicle may include an autonomous or semi-autonomous control system for controlling operation of the vehicle as the vehicle travels along a road toward a destination (e.g., the hospital destination received from the remote server). The communication system may communicate new or updated or continuous patient health data as the emergency vehicle travels toward the destination.

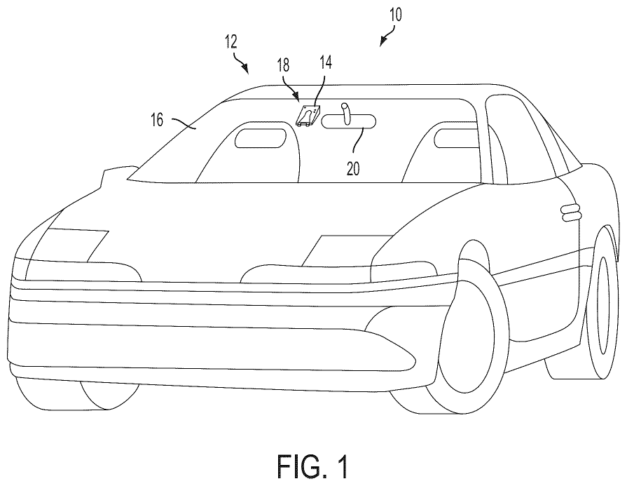

Referring now to the drawings and the illustrative embodiments depicted therein, a vehicle 10 equipped with the communication system 12 may include an electronics module 14 disposed at an inner portion of the vehicle, such as mounted to an interior surface of the vehicle like a headliner or in-cabin surface of a windshield 16 (FIG. 1). The electronics module 14 houses a control or electronic control unit (ECU) 18 having electronic circuitry and associated software. For example, the electronics module or the ECU 18 may include a wireless communication module (e.g., a transmitting and/or receiving antenna) so that the communication system 12 may wirelessly communicate with the remote server (e.g., via a transmitting and/or receiving antenna in communication with the remote server). As discussed further below, the vehicle 10 may be equipped with one or more sensors, such as one or more cameras disposed at the vehicle (e.g., a forward viewing camera disposed at the electronics module 14 and viewing through the windshield of the vehicle, or a driver monitoring camera viewing a head region of the driver of the vehicle, and/or a plurality of exterior cameras having a respective camera disposed at the front, rear and sides (such as at exterior rearview mirrors of the vehicle) and/or the like). Thus, the electronic circuitry may include a data processor or image processor that is operable to process image data captured by the camera or cameras, whereby the ECU may detect or determine presence of objects or the like and/or the system provide displayed images at a display device 20 for viewing by the driver of the vehicle (which may be part of or incorporated in or at an interior rearview mirror assembly of the vehicle, the control and/or the display device may be disposed elsewhere at or in the vehicle). The data transfer or signal communication from the camera to the ECU may comprise any suitable data or communication link, such as a vehicle network bus or the like of the equipped vehicle.

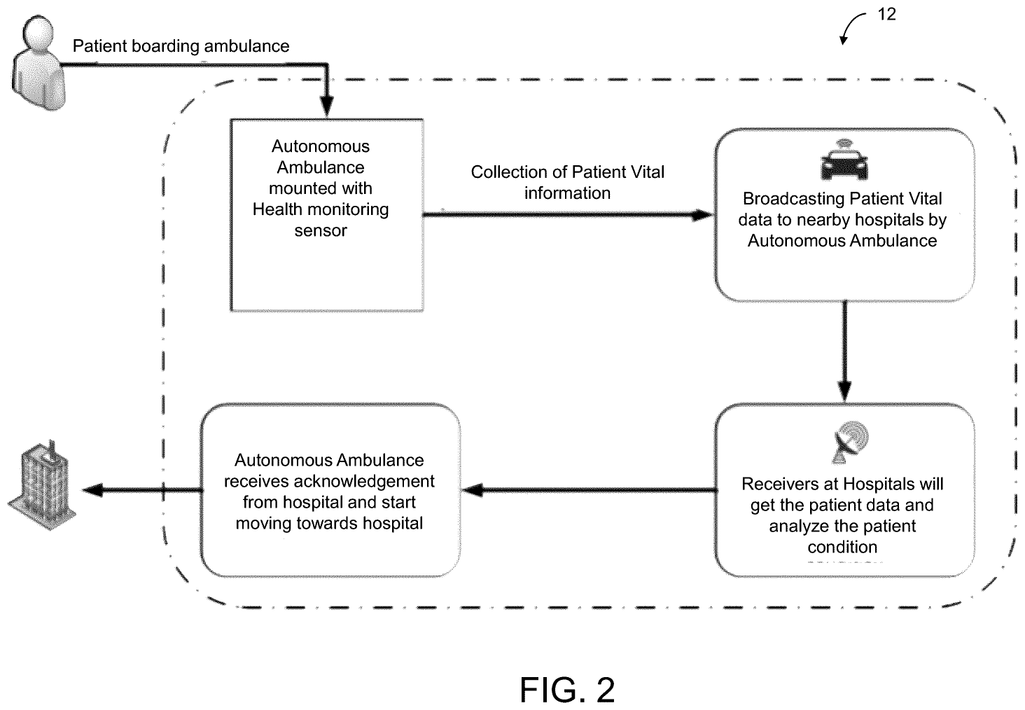

As shown in FIG. 2, the vehicle (such as an ambulance) may have health monitoring sensors mounted in the ambulance and configured to capture sensor data representative of a patient's vital information or health condition (such as blood pressure, glucose, accelerometer, body temperature, blood oxygen saturation (SpO2), pulse, patient risk factor, brain mapping information, and the like). The communication system 12 communicates the vital information to the remote server and receives communications from the remote server based on the communicated vital information. For example, the remote server may comprise or be in communication with receivers at one or more hospitals and personnel (e.g., doctors, nurses, and other medical professionals) or systems at the respective hospitals may analyze the vital information to determine whether the particular hospital is equipped to accept and properly treat the patient. The communication system then receives an acknowledgement that a particular hospital is equipped to accept the patient, and communicates a signal to a driving assist system of the vehicle to route the vehicle toward that particular hospital. Optionally, medical personnel operating the ambulance (e.g., emergency medical technicians) may manually input the patient's vital information for communicating to the remote server.

The vehicle may be an autonomous or semi-autonomously controlled vehicle, where the driving assist system at least partially controls operation (e.g., steering and/or acceleration and braking) of the vehicle as the vehicle travels along the road. Thus, the driving assist system may automatically begin moving the vehicle toward the accepted hospital after receiving the signal from the remote server. While on the way to the hospital, the autonomous ambulance can also communicate with traffic signals and initiate traffic clearance along the travel path.

While the vehicle travels toward the hospital, the system may continuously broadcast the patient's vital information to the nearby hospitals as the vehicle travels so that a monitoring team at the hospital can continuously analyze the patient's vitals and be prepared with an appropriate emergency response.

For example, soon after boarding a critical patient into the ambulance (or other emergency vehicle), the health monitoring sensor may be positioned to capture health data about the patient, such as via attaching the sensor to the patient's hands, wrist, chest, head, or other suitable bodily location. The ambulance continuously broadcasts the patient's vital information captured by the health monitoring sensor to a remote server in communication with receivers placed in nearby hospitals. The hospitals will receive the data and decide if they have sufficient facilities to handle the patient's condition. If the hospital agrees to take the patient's case, they will send an acceptance request to the ambulance. Thus, the vehicle may start travelling toward the accepted hospital. Control of the travel direction of the ambulance (e.g., to one hospital over another) may be adjusted by the system based on the severity of the patient's condition.

In other words, the vehicular communication or control system 12 is equipped at a vehicle that may be used to transport occupants experiencing health emergencies. One or more health monitors or sensors are disposed at the vehicle and operable to collect health data corresponding to the one or more occupants within the vehicle. For example, the sensors capture sensor data representative of the occupant's body temperature, heart rate, blood pressure, blood oxygen level, blood glucose level, and the like. The system wirelessly transmits signals to the remote system based on the captured sensor data and a current geographic location of the vehicle. For example, the system may transmit the captured data or a summary of the captured data (e.g., indicating captured data that is abnormal or indicative of a health condition or concern or issue), such as a signal indicating a level of criticality corresponding to the occupant's health condition. The system may wirelessly transmit the signal to the remote system, such as via a cellular network, an internet connection, and the like. Based on the signals received from the vehicular system, the remote system transmits a signal to the vehicular system that indicates a target destination for the vehicle. For example, the remote system determines a hospital destination for the occupant, such as a nearest hospital, a hospital best suited to treat the occupant's condition, a hospital having availability or capacity to treat the occupant, a hospital requesting to treat the patient, and the like. Based on the signal received from the remote system, the vehicular system determines a navigational route for the vehicle to follow to reach the target destination and may optionally control operation of the vehicle as the vehicle travels along the navigational route toward the target destination.

Optionally, the remote system may transmit a plurality of hospital destinations to the vehicle and the system may determine the target destination from the plurality of hospitals. For example, the vehicular system may determine the target destination based on a proximity of the vehicle to the target destination (e.g., the system may select the closest hospital or the shortest route time to the hospital, and may take into account current traffic conditions along the routes to different hospitals), a capability of the hospital to treat the occupant's health condition (e.g., the system may select a hospital with an expertise or focus on the health condition), an availability of the hospital to treat and/or admit the occupant (e.g., the system may select a hospital with a shorter wait time or vacant rooms over a hospital with a long emergency wait time or no vacant rooms), and the like.

The system at the autonomous ambulance procures patient details and populates the patient data to nearby hospitals. They system may broadcast the patient data to all hospitals and provide online monitoring of the patient by systems at the hospitals. Further, the vehicle may perform self-driving toward confirmed hospitals that can provide required medical attention.

Thus, use of the communication system may avoid or reduce the chance of death while a patient is being transported to a hospital. That is, some studies show that almost 20 percent of deaths (that occur in route to the hospital or upon arrival at the hospital) occur at least in part due to improper communication between ambulances and hospitals. Not all hospitals are equipped to handle all conditions for patient's received in emergencies. This may result in the patient being transferred again to a different hospital. The communication system may avoid or reduce the number of deaths that occur due to delays in responding to patient condition while being transferred to hospitals as, while the patient is being transported, a hospital with sufficient infrastructure can analyze the patient's condition and communicate with the communication system at the ambulance to ensure the patient is transported to the proper hospital and treated on time. Furthermore, a network of communication is created between the patient, the ambulance (e.g., an ambulance that is autonomously driven along the road), and hospitals. This feature may be implemented in ambulances or any health response vehicles.

Typically an autonomous vehicle would be equipped with a suite of sensors, including multiple machine vision cameras deployed at the front, sides and rear of the vehicle, multiple radar sensors deployed at the front, sides and rear of the vehicle, and/or multiple lidar sensors deployed at the front, sides and rear of the vehicle. Typically, such an autonomous vehicle will also have wireless two way communication with other vehicles or infrastructure, such as via a car2car (V2V) or car2x communication system.

The system may communicate with the remote server (e.g., a transmitting and/or receiving antenna in communication with the remote server) or other systems, such as via a vehicle-to-vehicle communication system or a vehicle-to-infrastructure communication system or the like. Such car2car or vehicle to vehicle (V2V) and vehicle-to-infrastructure (car2X or V2X or V2I or a 4G or 5G broadband cellular network) technology provides for communication between vehicles and/or infrastructure based on information provided by one or more vehicles and/or information provided by a remote server or the like. Such vehicle communication systems may utilize aspects of the systems described in U.S. Pat. Nos. 10,819,943; 9,555,736; 6,690,268; 6,693,517 and/or 7,580,795, and/or U.S. Publication Nos. US-2014-0375476; US-2014-0218529; US-2013-0222592; US-2012-0218412; US-2012-0062743; US-2015-0251599; US-2015-0158499; US-2015-0124096; US-2015-0352953; US-2016-0036917 and/or US-2016-0210853, which are hereby incorporated herein by reference in their entireties.

Changes and modifications in the specifically described embodiments can be carried out without departing from the principles of the invention, which is intended to be limited only by the scope of the appended claims, as interpreted according to the principles of patent law including the doctrine of equivalents.

1. A vehicular control system, the vehicular control system comprising:
an electronic control unit (ECU) disposed at a vehicle equipped with the vehicular control system, wherein the ECU comprises electronic circuitry and associated software;
wherein the vehicular control system is in wireless communication with a remote server that is remote from the vehicle;
wherein a health monitoring sensor is disposed at the vehicle, and wherein the health monitoring sensor captures sensor data representative of a health condition of an occupant of the vehicle;
wherein the vehicular control system, based on processing at the ECU of the captured sensor data, wirelessly transmits a first signal to the remote server, and wherein the first signal is based at least in part on the captured sensor data;
wherein, responsive to receiving the first signal from the vehicular control system, the remote server wirelessly transmits a second signal to the vehicular control system, and wherein the second signal comprises at least one destination suitable to treat the health condition of the occupant of the vehicle;
wherein the vehicular control system, based at least in part on the second signal, selects a target destination from the at least one destination; and
wherein the vehicular control system determines a path of travel of the vehicle between the vehicle's current geographic location and the target destination's geographic location, and wherein the vehicular control system at least partially controls operation of the vehicle as the vehicle travels along the path of travel toward the target destination's geographic location.

2. The vehicular control system of claim 1, wherein the electronic circuitry of the ECU comprises a wireless communication module that is operable to wirelessly communicate with the remote server.

3. The vehicular control system of claim 1, wherein the first signal comprises the captured sensor data.

4. The vehicular control system of claim 1, wherein the first signal comprises the vehicle's current geographic location.

5. The vehicular control system of claim 1, wherein the remote server determines the at least one destination suitable to treat the health condition of the occupant of the vehicle from a plurality of medical facilities based at least in part on the first signal.

6. The vehicular control system of claim 5, wherein the at least one destination suitable to treat the health condition of the occupant of the vehicle comprises one or more medical facilities of the plurality of medical facilities determined by the remote server to be capable to treat the health condition of the occupant of the vehicle.

7. The vehicular control system of claim 5, wherein the at least one destination suitable to treat the health condition of the occupant of the vehicle comprises one or more medical facilities of the plurality of medical facilities determined by the remote server to be available to treat the health condition of the occupant of the vehicle.

8. The vehicular control system of claim 5, wherein the first signal comprises the vehicle's current geographic location, and wherein the at least one destination suitable to treat the health condition of the occupant of the vehicle comprises one or more medical facilities of the plurality of medical facilities determined by the remote server to be located within a threshold distance of the vehicle's current geographic location.

9. The vehicular control system of claim 1, wherein the second signal comprises a plurality of destinations suitable to treat the health condition of the occupant of the vehicle.

10. The vehicular control system of claim 9, wherein the vehicular control system selects the target destination from the plurality of destinations suitable to treat the health condition of the occupant of the vehicle based at least in part on the second signal and the captured sensor data.

11. The vehicular control system of claim 10, wherein the target destination is selected based at least in part on a distance from the vehicle's current geographic location and the target destination's geographic location.

12. The vehicular control system of claim 10, wherein the target destination is selected based at least in part on a capability of the target destination to treat the health condition of the occupant of the vehicle.

13. The vehicular control system of claim 10, wherein the target destination is selected based at least in part on an availability of the target destination to treat the health condition of the occupant of the vehicle.

14. The vehicular control system of claim 1, wherein the vehicular control system autonomously controls operation of the vehicle as the vehicle travels along the path of travel toward the target destination's geographic location.

15. The vehicular control system of claim 1, wherein the target destination's geographic location comprises a medical facility.

16. The vehicular control system of claim 1, wherein the vehicle comprises an emergency services vehicle.

17. The vehicular control system of claim 1, wherein the occupant is a driver of the vehicle.

18. The vehicular control system of claim 1, wherein the occupant is a passenger of the vehicle.

19. A vehicular control system, the vehicular control system comprising:
an electronic control unit (ECU) disposed at a vehicle equipped with the vehicular control system, wherein the ECU comprises electronic circuitry and associated software;
wherein the vehicular control system is in wireless communication with a remote server that is remote from the vehicle;
wherein a health monitoring sensor is disposed at the vehicle, and wherein the health monitoring sensor captures sensor data representative of a health condition of an occupant of the vehicle;
wherein the vehicular control system, based on processing at the ECU of the captured sensor data, wirelessly transmits a first signal to the remote server, and wherein the first signal is based at least in part on the captured sensor data;
wherein, responsive to receiving the first signal from the vehicular control system, the remote server wirelessly transmits a second signal to the vehicular control system, and wherein the second signal comprises a plurality of destinations suitable to treat the health condition of the occupant of the vehicle;
wherein the plurality of destinations comprises a plurality of medical facilities;
wherein the vehicular control system, based at least in part on the second signal and the captured sensor data, selects a target destination from the plurality of medical facilities; and
wherein the vehicular control system determines a path of travel of the vehicle between the vehicle's current geographic location and the target destination's geographic location, and wherein the vehicular control system at least partially controls operation of the vehicle as the vehicle travels along the path of travel toward the target destination's geographic location.

20. The vehicular control system of claim 19, wherein the remote server determines the plurality of medical facilities suitable to treat the health condition of the occupant of the vehicle based at least in part on the first signal.

21. The vehicular control system of claim 20, wherein the plurality of medical facilities suitable to treat the health condition of the occupant of the vehicle comprises medical facilities determined by the remote server to be capable to treat the health condition of the occupant of the vehicle.

22. The vehicular control system of claim 20, wherein the plurality of medical facilities suitable to treat the health condition of the occupant of the vehicle comprises medical facilities determined by the remote server to be available to treat the health condition of the occupant of the vehicle.

23. The vehicular control system of claim 20, wherein the first signal comprises the vehicle's current geographic location, and wherein the plurality of medical facilities suitable to treat the health condition of the occupant of the vehicle comprises medical facilities determined by the remote server to be located within a threshold distance of the vehicle's current geographic location.

24. The vehicular control system of claim 19, wherein the target destination is selected based at least in part on a distance from the vehicle's current geographic location and the target destination's geographic location.

25. The vehicular control system of claim 19, wherein the target destination is selected based at least in part on a capability of the target destination to treat the health condition of the occupant of the vehicle.

26. The vehicular control system of claim 19, wherein the target destination is selected based at least in part on an availability of the target destination to treat the health condition of the occupant of the vehicle.

27. A vehicular control system, the vehicular control system comprising:
an electronic control unit (ECU) disposed at a vehicle equipped with the vehicular control system, wherein the ECU comprises electronic circuitry and associated software;
wherein the vehicular control system is in wireless communication with a remote server that is remote from the vehicle;
wherein a health monitoring sensor is disposed at the vehicle, and wherein the health monitoring sensor captures sensor data representative of a health condition of an occupant of the vehicle;
wherein the vehicular control system, based on processing at the ECU of the captured sensor data, wirelessly transmits a first signal to the remote server, and wherein the first signal is based at least in part on the captured sensor data;
wherein, responsive to receiving the first signal from the vehicular control system, the remote server wirelessly transmits a second signal to the vehicular control system, and wherein the second signal comprises a target destination suitable to treat the health condition of the occupant of the vehicle;
wherein the remote server selects the target destination suitable to treat the health condition of the occupant of the vehicle from a plurality of medical facilities based at least in part on the first signal; and
wherein the vehicular control system determines a path of travel of the vehicle between the vehicle's current geographic location and the target destination's geographic location, and wherein the vehicular control system at least partially controls operation of the vehicle as the vehicle travels along the path of travel toward the target destination's geographic location.

28. The vehicular control system of claim 27, wherein the first signal comprises the captured sensor data.

29. The vehicular control system of claim 27, wherein the first signal comprises the vehicle's current geographic location.

30. The vehicular control system of claim 27, wherein the target destination suitable to treat the health condition of the occupant of the vehicle comprises one medical facility of the plurality of medical facilities determined by the remote server to be capable to treat the health condition of the occupant of the vehicle.

31. The vehicular control system of claim 27, wherein the target destination suitable to treat the health condition of the occupant of the vehicle comprises one medical facility of the plurality of medical facilities determined by the remote server to be available to treat the health condition of the occupant of the vehicle.

32. The vehicular control system of claim 27, wherein the first signal comprises the vehicle's current geographic location, and wherein the target destination suitable to treat the health condition of the occupant of the vehicle comprises one medical facility of the plurality of medical facilities determined by the remote server to be located within a threshold distance of the vehicle's current geographic location.

33. The vehicular control system of claim 27, wherein the vehicular control system autonomously controls operation of the vehicle as the vehicle travels along the path of travel toward the target destination's geographic location.
